(19) United States
(12) Patent Application Publication
(10) Pub. No.: US 2005/0176936 A1
(43) Pub. Date: Aug. 11, 2005

(54) Method for obtaining a protein isolate and a fibre fraction from a parent substance containing fibres and protein

(76) Inventor: Waldemar Neumuller, Gottingen (DE)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/516,187

(22) PCT Filed: May 9, 2003
(86) PCT No.: PCT/EP03/04867

(30) Foreign Application Priority Data
May 31, 2002 (DE) 102242577

(51) Int. Cl. C07K 1/10; A61K 35/78

(52) U.S. Cl. 530/412; 424/725

(57) ABSTRACT

The invention relates to a method for obtaining a protein isolate and a fibre fraction from a parent substance containing fibres and protein. Said method comprises the following steps: conversion of the particles of the parent substance into an aqueous suspension; exertion of shear forces, which mechanically mill the particles in the suspension, whilst the fibres in the particles swell as a result of water absorption; monitoring of the pH value of the suspension during the swelling process of the fibres and maintenance of the pH value within a pH range that does not deviate from the natural pH value of the untreated parent substance by more than 2, optionally by the addition of acids and/or lyes; subsequent increase of the pH value of the suspension containing the swollen fibres to the alkaline range in order to release proteins that adhere to the fibres into an alkaline solution, whereby the particles containing the swollen fibres are finely distributed with an average particle size of between 10 and 150 ?m in a mass-weighted average; and isolation of one of the liquid phases containing the extracted proteins from the solids that are suspended in said phase and comprise the fibre fraction.

The invention relates to a method for obtaining a protein isolate and a fiber fraction from a fibrous and proteinaceous starting substance.

Plant protein sources are at the center of many considerations on covering an internationally increasing demand for functional proteins and as a supplier of functional dietary fibers. The center of the interest to date here was generally only in the protein fraction, that is to say in obtaining a protein isolate. The accompanying fibers have been considered solely as residues of the protein isolate production and not as a separate material of value. For a certain time, however, the fibers have also found greater interest, since they are distinguished by a high water-binding capacity. Such a water-binding capacity is a precondition for use as dietary fiber. A fiber fraction produced in protein isolate production, however, can only be used as dietary fiber when it has an adequate particle structure and good sensory properties. There is an interaction here between the particle structure and the sensory properties of the fiber fraction. When it is ensured that the fibers retain a coarse structure so that they can be separated off from extracted proteins by means of conventional centrifugation techniques, the capillaries of the fibers retain proteins and accompanying materials, which impair the sensory properties of the fibers. Typically, to separate off the fibers from dissolved proteins by means of centrifuging, particles having a size of from 200 to 600 μm are selected which are also termed grits. The proteins are typically extracted from the starting substance using an alkaline solvent. After separating off the alkaline solution from the fiber function, the proteins are precipitated at their isoelectric point by addition of acid. This precipitation is to a certain extent selective toward other soluble accompanying materials. In connection with the proteins and accompanying materials remaining in the capillaries of the fibers, it has to be taken into account that the effective uptake volume of the fibers for these substances is increased by a swelling of the fibers.

The apparatuses used for industrial centrifugation are known as decanters. A characteristic of all decanters is that, although they can process high solid concentrations, a part of their separation efficiency is lost due to air introduction. Such an air introduction is a problem additionally because the atmospheric oxygen, under the conditions present in alkaline extraction, readily leads to an oxidation of unsaturated fatty acids or the phenols always present in plant products. That is to say autoxidation takes place which leads to an impairment of the sensory aspects of product quality. This reaction is assumed, for example, to be the origin for the development of what is termed “beany taste” of the soy proteins. To reduce air incorporation in decanters and to maintain the separation efficiency, antifoams are frequently added to the suspensions to be centrifuged. These cause a reduction in the surface tension, and thus a lower foam formation. Their use, however, ultimately leads to the fact that the fibers separated off in the centrifugation, in addition to the unwanted constituents typical of the product and the oxidation products formed during the extraction, also still comprise the added antifoams. The fibers which are then dried are therefore not usable as dietary fibers, or only usable with great restrictions, since they have very strong characteristic taste and a sandy structure, the sandy structure also not being removable by finer grinding of the fibers.

However, with particle sizes becoming smaller, the ability of the fibers to be separated from the dissolved proteins by centrifugation decreases. In addition, at very small particle sizes, the water binding capacity of the fibers is lost.

For example, U.S. Pat. No. 6,284,292 has proposed counteracting the disadvantages of the strong swelling and the binding of the various substances to the fibers by breaking up the fiber structure using enzymes. Enzymes are used which attack the entire carbohydrate matrix, that is also cellulose fibers important for isolating dietary fibers. In the known processes the enzymes also reduce the viscosity of the suspension during the alkaline extraction. Thus the subsequent ability of the dissolved proteins to be separated from the solids by ultrafiltration is improved.

EP 0 479 596 A1 discloses supporting the alkaline extraction of the proteins by using protein- and carbohydrate-cleaving enzymes. The associated decrease in viscosity permits working at relatively high solids concentrations. The proteins are separated from the solids here by centrifugation.

EP 0 700 641 A2 discloses a purification of previously coagulated proteins by an alcohol extraction. The purified particles are then taken up in water and mechanically comminuted by shearing forces to from 1 to 14 μm. The comminuted suspension is subjected to an alkaline extraction in which proteases are additionally used, in order to bring the protein size below 8000 g/mol, at which separation by centrifugation is no longer possible.

The classic method of separating a protein isolate from a fiber fraction by alkaline extraction and subsequent centrifugation is described, for example, in EP 0 859 553 B1, WO 00/49887 or WO 99/17619. The resultant fiber fraction is burdened with the above-described disadvantages and can only be used as a dietary fiber with restrictions.

U.S. Pat. No. 5,508,172 discloses a method for the enzymatic modification of dietary fiber material, in particular a soybean fiber material, in which the sensory properties including the sandiness are improved by the fibers being hydrolyzed in an aqueous suspension using a mixture of a cellulase and a carbohydrase. The extent of the hydrolysis is to be selected so that it is sufficient to improve the sensory properties of the fiber material without significantly decreasing the proportion of dietary fibers.

The object underlying the invention is to demonstrate a method which is capable of simultaneously obtaining a protein isolate and a fiber fraction from a fibrous and proteinaceous starting substance, with, in addition to the protein isolate, the fiber fraction also being obtained in a quality which permits direct use in foods. In particular, the fiber fraction is to have a high water-binding capacity, but nevertheless is to be of neutral taste.

According to the invention this object is achieved by a method for obtaining a protein isolate and a fiber fraction from a fibrous and proteinaceous starting substance having the features of patent claim 1.

Advantageous embodiments of the inventive method are described in subclaims 2 to 20.

In the novel method, the surface area of the particles is increased before the alkaline extraction of the proteins. This increase in surface area occurs owing to the combination of a swelling of the fibers in the particles with shearing forces which act on the particles and mechanically comminute them. The particles are comminuted in a defined manner so that, by modern industrial centrifugation techniques, after the alkaline extraction of the proteins, separation of the undissolved fibers is still possible by centrifugation. The smallest particle sizes which are still usable for the separation by centrifugation are at the same time large enough that the water-binding capacity of the fiber fraction as dietary fiber is not impaired. The retention capacity of proteins and other accompanying materials in the capillaries of the fibers is already reduced, however. Since, at the same time, by monitoring of the pH of the suspension during the swelling of the fibers, denaturation of the proteins is prevented, these do not bind in a stronger manner to the fibers. Preference is given to the swelling of the proteins taking place under the action of shearing forces at a pH which remains around 0.6 above the isoelectric point of the proteins. Below this pH, a decrease in denaturation occurs, which binds the proteins more strongly into and onto the fibers. If, in contrast, the pH is increased much further, a structural change of the fibers occurs in which a tendency for association to the dissolved proteins exists, which is likewise disadvantageous for the desired separation of the fibers from the proteins. In all cases it is necessary to perform the swelling of the fibers while observing a pH range which deviates by no more than +/−2 from the natural pH of the untreated starting substance. Preference is given to a narrower range of less than +/−1.0, in order to prevent an unwanted denaturation of the proteins and such a structural change of the fibers. A pH range in which the novel method functions well is between 5.0 and 6.5. The shearing forces acting on the particles during the swelling of the fibers markedly improve the extractability and separability of the proteins from the fibers. This appears to be due to a structural change which goes beyond the mechanical comminution of the particles. The shearing forces during the swelling of the fibers can be applied by all known types of homogenization techniques, for example colloid mills, ultrasound, rotor-stator systems, high-pressure disintegration etc. It is important that the shearing forces have sufficient magnitude which is typically documented by the particles in the suspension actually being comminuted under the action of the shearing forces, even if they have a starting particle size of 150 μm or less. The centrifugation techniques to be used in the novel method are to be matched to the fine distribution of the particles. A suitable technique is, for example, a commercially conventional decanter, to which is connected downstream a separator, the decanter separating off the major fraction of the solids, so that the separator can concentrate on separating off the smaller particles of the solids, and is only charged with small solid quantities. The use of what is termed a prodecanter from the company Flottweg, Germany, is particularly advantageous. This apparatus builds up a centrifugal field similar to a separator and is nevertheless suitable for continuously processing relatively high solids concentrations.

The temperature of the suspension is preferably maintained in a range of from 40 to 60° C. during the swelling. Particular preference is given to the temperature range of from 45 to 55° C., that is to say about 50° C. The time period of swelling is to be adjusted via the result of the swelling. The swelling of the fibers should have achieved at least more than 50% of the maximum water absorption of the fibers before the pH of the suspension is elevated to the alkaline range. Preferably, the swelling is more than 75% of the maximum swelling.

This is achieved under the action of the shearing forces during swelling, typically over a period of from 10 to 60 min. Generally, about 20 min, that is to say from 15 to 25 min, are suitable for a very good swelling result.

During the swelling, the particles should preferably be comminuted by the shearing forces to a fine distribution having a mean particle size of from 80 to 100 μm on a weight average. This can then be followed by the use of further shearing forces which act on the particle having the already swollen fibers, in which case these shearing forces should be greater than the shearing forces during the swelling. Such greater shearing forces can be applied, for example, by high-pressure disintegration via a pressure difference of at least 80 bar, preferably of at least 100 bar. As a result of the total shearing forces, the particles having the swollen fibers are preferably comminuted to a mean particle size of below 80 μm on a weight average. It is ideal if the particles, before the pH of the suspension is elevated to the alkaline range, have particle sizes in the range from 10 to 100 μm. This should be the case for at least 80% by weight, preferably for at least 90% by weight, of the particles.

A good indication of successful pretreatment of the fibrous and proteinaceous starting substance in the novel method is if the volume of the 10% by weight dry matter suspension which can be sedimented at 1000×g within 5 min is elevated by the entire shear forces to from 20 to 60% by volume, preferably from 30 to 60% by volume.

The novel method is very gentle to proteins, since elevated temperatures can be dispensed with not only in the pretreatment but also in the alkaline extraction.

However a disadvantage is considered to be that natural enzymes present in the starting substance such as phenol oxidases and trypsin inhibitors are only slightly inactivated. At the same time, the increased surface area of the particles also makes the lipid and phenols associated with the proteins more susceptible to oxidative attack in the alkaline extraction. However, this can be prevented by carrying out the alkaline extraction in the presence of a reducing agent. Sodium sulfite (Na₂SO₃) proves to be particularly suitable here. The reducing agent stops the autoxidation and prevents the unwanted reaction products. The concentration in use of the sodium sulfite is typically from 0.25 to 2 g per kg of protein.

Because of the fine distribution of the particles in the suspension, the time period of the alkaline extraction in the novel method can also be reduced markedly below 30 min, as it necessary in the extraction of coarser particles. In the preferred alkaline range of pH from 9 to 10, extraction times of at most 15 min can be realized. These short extraction times also reduce the tendency to autoxidation.

To achieve the desired fine distribution of the particles in the preferred range of below 80 μm, carbohydrate-cleaving enzymes can also be used in the novel method. Those which have proved to be suitable are hemicellulases, pectinases, glucosidases and amylases. Cellulases, in contrast, are unsuitable for use in the novel method. They lead rather to an impairment in the structure of the fibers, which impairs their separability from the proteins and their use as dietary fiber. The enzymes are preferably added to the particles before the further shearing forces are applied, and then an enzyme reaction time of from 30 to 120 min is allowed. The enzymes open the fiber structure so that the desired fine distribution of the particles is achieved with reduced shearing force work.

The novel method is extremely effective overall not only with respect to yield, but also the functionality of the products produced. To comply with microbiological requirement of the food industry on the products of the method, not only the protein isolate obtained, but also the fiber fraction, can be pasteurized. Pasteurization at the end of the method has the advantage here that the product is free from unwanted accompanying materials during the heating, which stops any thermally excited interaction of the accompanying materials with the constituents. The possibly existing disadvantage of a heavier contamination of the apparatuses used to carry out the method over relatively long production times is opposed by the rapidity of the extraction, the improved functionality of the products, and the increased creation of value by obtaining protein isolate and fibers as dietary fiber. However, if the starting substance is a heavily contaminated material, a thermal treatment is also possible at the start of the method. This should at any rate be performed before swelling of the fibers with action of the shearing forces, since heating after the swelling causes particle enlargement which is then uncontrolled.

The already fundamentally good yield of the novel method can be improved by a multistage alkaline extraction in which the solids comprising the fiber fraction, from which the liquid phase comprising the extracted proteins has been separated, is extracted at least one further time in alkaline conditions and in which a further liquid phase comprising the thus extracted proteins is separated from the remaining solids. This further liquid phase can be recirculated to the extraction stage preceding the additional extraction stage. Thus sodium sulfite, which is only added in the second extraction stage of a plurality of extraction stages is also present and active at a certain concentration in the extraction stage. At the same time, a sodium sulfite concentration increasing from the first to the second extraction stage also corresponds to an increasing risk of autoxidation of the proteins, as occurs if stronger alkali conditions are present in the second extraction stage than in the first, in order still to detach from the solids proteins which have not yet passed into solution.

A multistage alkaline protein extraction is disclosed in principle by DE 199 07 725 A1. Here, however, at least one protease is always conducted in countercurrent to the solid. In the novel method described here, no protease at all need be used. Nevertheless, a protease can further increase the yield of the novel method and thus the purification of the fiber fraction to free it from the adhering proteins. Starting from a three-stage protein extraction, for example, at least one protease can be added in the third extraction stage in order to bring into solution the proteases which cannot be detached from the solids solely under alkali conditions. Frequently, the proteins which are particularly strongly bound to the fibers are hydrophobic proteins. In detail, the overflow of the third separation, after its desalination, can be dried directly, or fed to the overflow of the first separation or a protein curd precipitated therefrom, instead of feeding it to a preceding protein extraction, so that the enzyme treatment remains restricted to the last extraction stage and the protease does not lead to unwanted hydrolysis of the proteins in the preceding extraction stages.

In concrete terms, hydrophobic proteins which are strongly bound to the fiber matrix can also be hydrolyzed in the last extraction stage by adding a neutral or alkaline protease at a pH of from 9 to 11, preferably approximately 10, to the extent that they can be separated from the fibers with the liquid phase. In the process, the still-bound proteins are cleaved into medium-chain to short-chain peptides. The pH of the solids dispersion decreases in parallel by 2 to 3 units. After a reaction time of 10 to 20 minutes, the dispersion is then adjusted with acid to a pH of approximately 6.5 and briefly heated to from 90 to 100° C. by steam injection, to inactivate the enzyme. After the dispersion is cooled to below 50° C., it is separated by centrifugation, after which the fiber fraction remaining as solids is substantially free from proteins which possibly trigger allergies.

The invention is discussed and described in more detail below with reference to examples; in the examples,

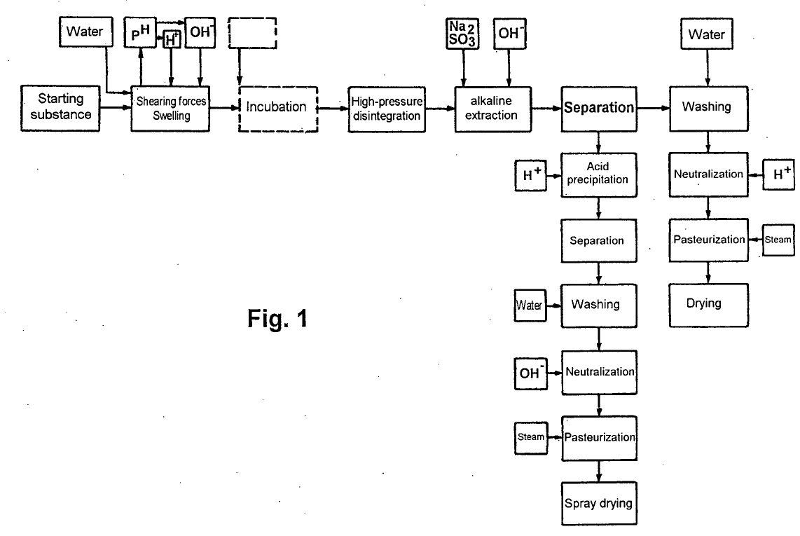

FIG. 1 shows the course of the novel method as a block diagram,

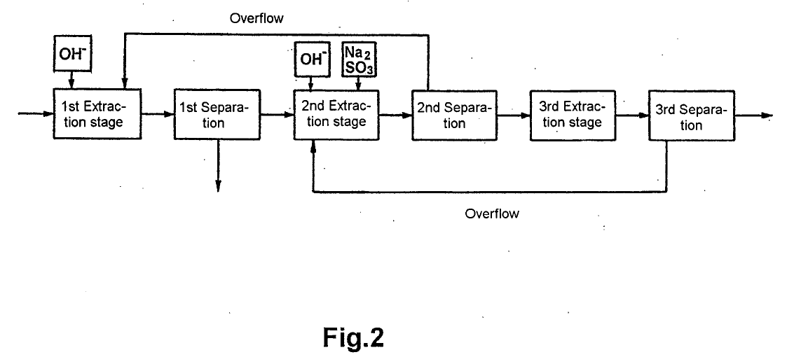

FIG. 2 shows a more detailed block diagram of the alkaline extraction and the separation in the process as according to FIG. 1.

In the process evident from FIGS. 1 and 2, a fibrous and proteinaceous starting substance is slurried with water under the action of shearing forces. This procedure takes about 20 min. In the procedure a comminution takes place with uptake of water. Depending the starting substance, acid (H⁺) or alkali (OH⁻) is added so that the pH of the resultant suspension remains above the isoelectric point of the protein. Ideally, this value should be between pH 5 and 6.5. The temperature of the suspension during the swelling is typically 50° C. An enzyme can then added to the suspension. In this case it is necessary to wait for an incubation of the suspension, that is to say an enzyme reaction time, depending on raw material, of from 30 to 120 min. The suspension is then homogenized by high-pressure disintegration, strong shearing forces acting on the particles in the suspension and further comminuting them. If there has been no preceding use of enzymes, the work of the shearing forces during the high-pressure disintegration must generally be greater than when an enzyme has been used.

Then, there is an alkaline extraction of the proteins from the comminuted particles of the starting substance having the swollen fibers. To prevent autoxidation, sodium sulfite dissolved in water is added so as to set a concentration of from 0.25 to 2 g, based on 1 kg of protein. For the alkaline extraction itself, alkali (OH⁻) is added to the suspension. The dissolved proteins are then separated from the undissolved solids using a prodecanter from Flottweg. The alkaline extraction can be in several stages here and take place in the countercurrent flow process, as is shown in more detail in FIG. 2. In this case, in three successive extraction stages with in each case subsequent separation, the pH and/or the temperature is increased at least once, to bring the proteins into solution under conditions becoming more aggressively alkaline. Here, in concrete terms, the alkali is added at the start of the first and second extraction stage and the sodium sulfite only at the start of the second extraction stage. The further extraction stages are supplied with alkali or sodium sulfite via the solids discharge of the preceding separation, or from the overflow of subsequent separation. In the case of the multistage extraction, the proteins are always exposed only to the alkaline conditions which are necessary for their dissolution. That is to say the readily soluble proteins are treated more mildly than the sparingly soluble proteins. Overall, the proteins are thus brought into solution as mildly as possible, that is to say with as few denaturations as possible. A multistage alkaline extraction having increasing strength of the alkaline conditions is disclosed by DE 199 07 725 A1. Here, however, in addition to the alkali, a protease is conducted in countercurrent. This protease is not necessary here in the novel method. If proteases are used here, this can take place, however, in a third extraction stage which is separated from the preceding extraction stages to the extent that the overflow of their separation is not recirculated to the preceding extraction stages. The proteases thus act selectively only in the third extraction stage, that is to say only on the proteins which have then not yet been brought into solution. This method variant is described in more detail by the following example 4. The extraction of the novel method is generally performed in a pH from 7 to 12, in particular from 9 to 10. The distribution of particle sizes offers an attack area for the alkali which is so large that an extraction time of in total less than 15 min can be achieved. An extraction time of approximately 10 min has proved to be sufficient. The shortening of the alkaline extraction markedly reduces the formation of toxic amino acids, for example lysinoalanine.

The separation according to FIG. 1 divides the mass stream into two parts. The left mass stream in FIG. 1 comprises the liquid phase from the separation. From this, by adding acid (H⁺), the proteins are precipitated at their isoelectric point, that is to say coagulated. No heating of the protein is required in the context of acid precipitation. The next step is to separate off the protein as protein curd from the remaining liquid phase. This is performed, just as in the separation of the solids from the dissolved proteins, either by a combination of a decanter and a separator, or by a prodecanter from Flottweg. Although other separation techniques, for example filtration, can be realized, they are scarcely economical. The protein curd obtained having a solids concentration of from 18% to 23%, that is to say approximately 20%, is washed with water. The wash water is separated off again. It is used to wash out the salts which have collected in the protein curd. The pH of the protein curd is then neutralized, more precisely in the range from 6.5 to 7. The neutralized protein curd can be pasteurized by steam injection. For this it must be heated to approximately 80° C. for a maximum of 2 min. The protein curd is then cooled again to below 50° C. Thereafter the protein isolate obtained can be spray dried. The functionality of the protein isolate corresponds, with respect to all customary properties, at least to the currently offered industry goods, with the additional advantage of a very high whipping ability and foam stability which goes significantly beyond the currently customary industrial standard. The fiber fraction which was separated off in the separation from the protein is washed with water. Then a neutralization with acid (H⁺) is performed. A pasteurization with steam is then performed in accordance with the pasteurization of the protein. Finally, the fibers are dried and, in the resultant form, are usable as dietary fiber.

Further details of the invention may be concluded from the following concrete working examples:

### EXAMPLE 1

Soybean flour of the type “White Flakes” (Cargill) is homogenized in water using a dissolver, so that the pH is adjusted to 5.5 and the dry matter to approximately 12%. The temperature of the homogenate is 50° C. After 15 min, the swollen homogenate is disintegrated using a high-pressure disintegrator at 100 bar. The sedimentable volume of a 10% strength solids solution increases from 25% to 45%. This homogenate is then set to a pH of 9 by sodium hydroxide solution with stirring. After a residence time of 10 min, the suspension is fed to a prodecanter at a solids concentration of from 8 to 12%. The solids discharge of the prodecanter is combined with water and a return of the subsequent extraction stage in such a manner that the solids content of the subsequent stage is approximately 6%. The pH is set to 9.5 and 0.5 g of sodium sulfite per kg of protein dissolved in water is added. Thereafter, centrifugation is performed again. This can be followed by third extraction stage. The pH of the fibrous fraction is established at from 8.5 to 9. By adding water, the pH is lowered to 8. If the addition of water alone should not suffice to achieve the required pH, it can be corrected with acid. On account of the following pasteurization and drying, the fiber fraction should be flowable, but not too watery. The outflow of the first separation is fed to a coagulation tank and set to pH 4.4 by hydrochloric acid. The protein curd forming is kept in suspension by intensive stirring, and centrifuged via a further prodecanter. Between precipitation and centrifugation there should be at least 10 min. The solids discharge which comprises the protein is resuspended with cold water, the water temperature establishing itself at 30° C. This solution is centrifuged again. The solids then arising are neutralized to pH 6.5 by means of sodium hydroxide solution and heated to 70° C. by means of steam injection for 30 sec. The medium is cooled in continuous flow to 50° C. and stored until drying by a spray tower. After evaluation of the resultant products, the following values were obtained before drying:

Protein isolate:

| | |
|---|---|
| Protein purity: | 92% |
| Yield: | 80% |
| Functionalities: | Standard values of industrial |
| | soybean isolates, having |
| | extremely pronounced foam- |
| | formation capacity. |
| Sensory properties: | Neutral taste |
| Color: | Creamy white |
| Protein solubility: | 95% |

Dietary fiber:

| | |
|---|---|
| Particle size: | <100 μm |
| Protein content: | <15% on MD |
| Water binding: | 600% |
| Color: | Creamy white |
| Sensory properties: | Neutral taste |

### EXAMPLE 2

GMO-free soybean flour of the “White Flakes” type (Solbar) is homogenized in water to give a dry matter content of 20%. The temperature of the suspension is 50° C. The pH is set to pH 5.8 using calcium chloride and hydrochloric acid. To this suspension is added a mixture of carbohydrate-cleaving enzymes (no cellulases) in a concentration of 0.4% on dry matter and the suspension is stirred over 90 min. After this enzyme reaction time, the suspension is homogenized using a high-pressure disintegrator at 150 bar (2000 psi). The sedimentable volume of a 10% strength solids solution increases from 20% to 50%. This homogenate is then set to pH 9 by means of sodium hydroxide solution under intensive stirring. After a residence time of 10 min, the suspension is fed to a prodecanter at a solids concentration of 10%. The solids discharge is combined with water and a return from the subsequent stage in such a manner that the solids content of the subsequent stage is approximately 6%. The pH is set to 10.5 and 0.5 g of sodium sulfite dissolved in water are added per kg of protein. Thereafter, centrifugation is performed. This can be followed by a third extraction stage. The pH of the fibrous fraction is established at from 8.5 to 9. By adding water, the pH is lowered to pH 8. If the addition of water alone should not suffice to achieve the required pH, it can be corrected with acid. On account of the subsequent pasteurization and drying, the fiber fraction should be flowable, but not too watery. Subsequently, the procedure of example 1 is followed. The isolate produced in this method is identical to the product from example 1. The dietary fiber shows a further improved water-binding capacity and improved mouth sensory properties.

### EXAMPLE 3

Soybean flour of the “Toasted Flakes” type (ADM) milled to 150 μm is suspended in water to give a dry matter content of approximately 20% and a pH of 5.5. The temperature of the suspension is 50° C. This suspension is admixed with 0.6% of an adapted enzyme mixture and incubated for 90 min with stirring. The suspension is then high-pressure disintegrated at 200 bar. The sedimentable volume of the 10% strength solids solution increases from 30 to 50%. This homogenate is then set to pH 10 using sodium hydroxide solution with intensive stirring. After a residence time of 12 min, the suspension is fed to a prodecanter. The solids discharge is combined with water and a return from the subsequent separation stage so that the solids content of the subsequent stage is approximately 12%. Before it is fed to the next centrifugation, the pH is set to pH 11 and 1.5 g of sodium sulfite per kg of protein dissolved in water are added. The second centrifugation can be followed by a third extraction stage. The pH of the fibrous fraction separated off establishes itself at 10. By addition of water and acid, the pH is reduced to pH 8. The outflow of the first prodecanter stage is fed to a coagulation tank and set to pH 4.4 by means of hydrochloric acid. The curd forming in the course of this is kept in suspension by intensive stirring and is again centrifuged via a further prodecanter. There are 10 min between precipitation and centrifugation. The solids discharge which comprises the protein is resuspended with cold water, the water temperature establishing itself at about 30° C. This suspension is centrifuged. The solids then arising are neutralized to pH 6.8 by means of sodium hydroxide solution and heated to 80° C. by means of steam injection. The medium is cooled in continuous flow to 50° C. after approximately 30 sec and is stored until drying by a spray tower. The resultant products have the following data:

Protein isolate:

| | |
|---|---|
| Protein purity: | 90% |
| Yield: | 76% |
| Functionalities: | Standard values of industrial |
| | soybean isolates, having |
| | extremely pronounced foam- |
| | formation capacity. |
| Sensory properties: | Neutral taste |
| Color: | Creamy white |
| Protein solubility: | 85% |

Dietary fiber:

| | |
|---|---|
| Particles: | <100 μm |
| Protein content: | <16% on MD |
| Water binding: | 400% |
| Color: | Creamy white |
| Sensory properties: | Neutral taste |

### EXAMPLE 4

Soybean flour of the “White Flakes” type (Cargill) is first treated as in example 1. In departure from the procedure in example 1, after the second centrifugation at the pH of 9.5, the pH of the solids discharge is set to 10 using aqueous alkali. The resultant suspension of the solids should, with good dispersibility, have a solids content of from 6 to 10%, and a temperature of from 40 to 50° C. To this dispersion is added at least one protease so that the concentration of enzyme is 1%, or 0.025 Anson units based on 1 kg of protein of the dispersion. After addition of the protease, the dispersion reacts for 20 minutes without pH correction. Within this time the pH falls to 7.5 to 8. The pH is then further reduced to 6.5 using acid, and the dispersion is heated to from 90 to 100° C. by steam injection for 45 seconds. After it has cooled to 50° C., the dispersion is centrifuged. The fiber fraction which is separated off can be fed directly to a drying stage, and it comprises a low-allergen dietary fiber. The overflow is collected separately and not returned to the previous extraction stage, but, for example, after desalting, likewise dried directly or, before its drying, fed to the protein curd which was precipitated from the overflow of the first centrifugation in accordance with example 1. The values below result from the latter procedure:

Protein isolate:

| | |
|---|---|
| Protein purity: | 91% |
| Yield: | 92% |
| Functionalities: | Standard values of industrial |
| | soybean isolates, having |
| | extremely pronounced foam- |
| | formation capacity. |
| Sensory properties: | Neutral taste |
| Color: | Creamy white |
| Protein solubility: | 98% |

Dietary fiber:

| | |
|---|---|
| Particle size: | <100 μm |
| Protein content: | <5% on MD |
| Water binding: | 600% |
| Color: | Creamy white |
| Sensory properties: | Neutral taste |

1. A method for obtaining a protein isolate and a fiber fraction from a fibrous and proteinaceous starting substance, comprising the steps:
converting particles of the starting substance into an aqueous suspension,
applying shearing forces which cause a mechanical comminution of the particles in the suspension over a period of at least 10 minutes, while the fibers in the particles swell owing to water uptake,
monitoring the pH of the suspension during the swelling of the fibers and maintaining the pH in a non-alkaline pH range which is above the isoelectric point of the proteins and deviates by no more than 2 from the natural pH of the untreated starting substance, if appropriate by adding acids and/or alkalis,
without a protease being present during the swelling of the fibers,
subsequently elevating the pH of the suspension having the swollen fibers to the alkaline range to bring proteins adhering to the fibers into solution under alkaline conditions, the particles having the swollen fibers having a fine distribution having a mean particle size of from 10 to 150 μm as weight average, and
separating by centrifugation a liquid phase comprising the extracted proteins from the solids suspended therein and comprising the fiber fraction.

2. The method of claim 1, wherein the pH of the suspension during the swelling of the fibers is maintained in a pH range which deviates by less that 1.0 from the natural pH of the untreated starting substance and is at least 0.5 above the isoelectric point of the proteins, preferably between 5.0 and 6.5.

3. The method of claim 1 or 2, wherein the temperature of the suspension during the swelling of the fibers is maintained in a range from 40 to 60° C., preferably from 45 to 55° C.

4. The method of one of claims 1 to 3, wherein the pH of the suspension is not elevated to the alkaline range before the swelling of the fibers has reached more than 50%, preferably more than 75%, of the maximum water absorption of the fibers.

5. The method of one of claims 1 to 4, wherein the shearing forces during the swelling of the fibers are applied over a period of up to 60 minutes, preferably from 15 to 25 minutes.

6. The method of one of claims 1 to 5, wherein the particles during the swelling are comminuted by the shearing forces to a fine distribution having a mean particle size of from 80 to 100 μm on a weight average.

7. The method of one of claims 1 to 6, wherein, before elevating the pH of the suspension to the alkaline range, further shearing forces are applied to the particles having the previously swollen fibers which shearing forces are greater than the shearing forces during the swelling.

8. The method of claim 7, wherein the greater shearing forces are applied by high-pressure disintegration via a pressure difference of at least 80 bar, preferably at least 100 bar.

9. The method of claims 7 or 8, wherein the particles having the swollen fibers are comminuted by the entire shearing forces to a weight-average mean particle size of less than 80 μm on a weight average.

10. The method of claim 9, wherein the particles having the swollen fibers are comminuted by the entire shearing forces to particle sizes of which at least 80% by weight of which, preferably at least 90% by weight, are between 10 and 100 μm.

11. The method of one of claims 7 to 10, wherein the volume of the 10% by weight dry matter suspension which can be sedimented at 1000×g within 5 minutes is elevated by the entire shearing forces to from 20 to 60% by volume, preferably from 30 to 60% by volume.

12. The method of one of claims 1 to 11, wherein the alkaline extraction of the proteins is carried out in the presence of sodium sulfite.

13. The method of claim 12, wherein concentration of the sodium sulfite is from 0.25 to 2 grams per kilogram of protein.

14. The method of one of claims 1 to 13, wherein the alkaline extraction is carried out at a pH of from 7 to 12, preferably from 9 to 10, within a maximum of 15 minutes.

15. The method of one of claims 7 to 10, wherein, before the application of the further shearing forces to the particles having the previously swollen fibers, at least one carbohydrate-cleaving cellulase-free enzyme is added to the suspension and an enzyme reaction time of from 30 to 120 minutes is allowed.

16. The method as claimed in one of claims 1 to 15, characterized in that the separated proteins and/or the fiber fraction is pasteurized.

17. The method of one of claims 1 to 16, wherein the solids comprising the fiber fraction from which the liquid phase comprising the extracted proteins has been separated are subjected to at least one further alkaline protein extraction with subsequent separation of a further liquid phase comprising the resultant extracted proteins.

18. The method of claim 17, wherein the further liquid phase separated after the further protein extraction is recirculated to the protein extraction preceding it.

19. The method of claim 12 or 13 and claim 18, wherein the sodium sulfite is added only in the second extraction stage of a plurality of extraction stages.

20. The method of one of claims 16 to 19, wherein, in a last protein extraction of a plurality of sequential protein extractions, at least one protease is used, the further liquid phase comprising the resultant extracted proteins not being recirculated to a preceding protein extraction.
